(19) United States
(12) Patent Application Publication
(10) Pub. No.: US 2006/0024368 A1
(43) Pub. Date: Feb. 2, 2006

(54) Compressed composite delivery system for release-rate modulation of bioactives

(76) Inventors: Reza Fassihi, Fort Washington, PA (US); Viness Pillay, Parktown (ZA)

Correspondence Address: RATNERPRESTIA, P O BOX 980, VALLEY FORGE, PA, 19482-0980, US

(21) Appl. No.: 10/903,267

(22) Filed: Jul. 30, 2004

(51) Int. Cl. A61K 9/24

(52) U.S. Cl. 424/473

(57) ABSTRACT

The invention is a delivery system comprising a first outer zone which partially surrounds an inner core, a second outer zone which also partially surrounds the core, and the outer zones together form a continuous heterogeneous layer fully surrounding the core. The delivery system is particularly suitable for orally administered multiple drug delivery or multiple rate delivery of biologically active ingredients to the gastrointestinal environment of humans or other animals.

### FIELD OF THE INVENTION

The invention relates to a delivery system for controlled, timed, release of biologically active ingredients. In particular, this invention relates to delivery systems for controlled delivery of biologically active substances that correspond to circadian and physiological variations.

### BACKGROUND OF THE INVENTION

In the past two decades emphasis has been placed on the development of oral drug delivery systems that provide zero order release kinetics. This has mainly been due to the recognized advantages of constant drug delivery over classic release patterns such as first order or “square root” kinetics. Essentially, maintaining strict control of the release characteristics so that a straight line release of drugs from the delivery system (that is, zero order release) is approximated, has been equated with and thought to provide approximately constant blood levels within the therapeutic range and provides a mechanism by which one may minimize many of the adverse effects of some drugs.

With the recognition of diurnal variations in physiologic processes and subsequent implications of chronopharmacokinetics, the need to provide a specific drug delivery pattern still remains a challenge from the perspective of both therapeutics-chronotherapeutics, and system design and development. Based on the appropriate timing for delivery of physiologically optimal drug concentrations, it has become apparent that zero order release either alone or combined with initial rapid drug release is not necessarily the most favorable profile for rate-controlled delivery of a drug regimen. To provide therapeutically more desirable drug levels which would accommodate both the diurnal requirement, that is, once-a-day or twice-a-day dosing, and maximum absorption during gastrointestinal transit, particularly as the dosage progresses into or enters the distal colon, a delivery system is required which is simple to manufacture using standard high speed tableting equipment and provides a combination of release conditions specific to the drug or drug combinations to be delivered, as well as to the period over which successive doses of the drug are to be delivered. Such a system must take into account several factors, including induction time and dosage level, desirable lag time/burst effect depending on where/when the initial dose is to be delivered, and the need for extended up-curving, zero order, biphasic or triphasic drug delivery. The initial slow release may correspond to a very high surface area and relatively short transit time in the stomach and small intestine. The absorption of drugs in this region is fast and complete for those drugs showing high permeability (i.e. F>0.7). On the other hand more rapid drug release may be desirable during the late time period when higher viscosity and low surface area of the large intestine could impose rate-limiting transport and absorption effects. If daily dosing is sought, the higher viscosity and low surface area of the large intestine and distal colon indicate an even higher rate of release in the period immediately preceding administration of the next dose of medication.

The concepts expressed in the preceding paragraph may be demonstrated by reference to FIG. 1, which illustrates the relationship between GI physiology, in terms of its viscosity, surface area, and drug transport rate into the blood, all as a function of time as the drug delivery system moves from the stomach, through the intestines and to the colon. Such diverse environmental conditions demonstrate the need for the drug release rate and pattern to be modulated so that they are in compliance with this pharmacodynamic/pharmacokinetic behavior.

Numerous approaches have been evaluated for providing lag time or steady-state drug release kinetics, including osmotic pump systems, triple-layer tablet designs, as well as recently reported hydrophilic devices. In general, existing technologies for compression-coated and layered tablets utilize either a concave cylindrical core on which a dry press coating procedure is applied or utilize a triple-layered configuration in which a core is sandwiched between two external layers in such a manner as to expose a peripheral edge of the core through which active ingredient(s) may be released. In triple-layer technologies, both lag time induction and zero order drug delivery can be achieved. This may essentially be due to controlling exposure of the surface area of the core or central layer to the infiltrating hydrating medium by programming the rate of barrier erosion. In monolithic designs, a lag phase is usually generated by coating a tablet with a pH-sensitive or slowly dissolving polymer. Although such complex rate-programmed systems may be capable of producing steady-state kinetics, with or without a lag phase, demonstration of up-curving, multi-phasic with variable release kinetics in response to certain circadian rhythms and overall gastrointestinal absorption still remains a challenge.

Thus, for example, Conte, U.S. Pat. No. 5,626,874 discloses a tri-layered tablet in which the active ingredient is contained in the central layer which is exposed above and below, to a barrier layer, but the outer periphery of the central layer is exposed to the infiltrating medium for release of the active ingredient. A variation of that design is shown in Fassihi, U.S. Pat. No. 5,783,212, incorporated herein by reference, in which there are two barrier layers comprising swellable hydrophilic polymers and a swellable central layer which contains the active ingredient. In this patent, drug release is achieved by swelling and water infiltration into all three layers followed by erosion of the swellable layers and release of the active ingredient from the exposed peripheral surface of the central drug containing layer. Both such systems are designed to achieve zero order or linear delivery over an extended period of time and to avoid significant induction lag time and burst effects. Therefore, a need exists for a drug delivery system that provides a drug delivery pattern adapted to specific physiological conditions and diurnal rhythms.

### SUMMARY OF THE INVENTION

The invention provides a delivery system for delivery of one or more bioactive agents in a multitude of patterns compatible with physiological and dosing requirements as discussed generally above. The delivery system comprises an outer layer comprising two heterogeneous barrier zones and an internal central core embedded in and fully surrounded by the outer layer. Through this design, rate-controlled heterogeneous erosion of either or both of the external zones and/or core and timed, rate-controlled release may be achieved. Control of the release process is therefore predominantly a function of system configuration, related swelling dynamics, floatation, and associated erosion of the zones comprising the external layer and/or internal core structure. This structure provides a degree of flexibility and adaptability that is not available with any known existing drug design system available for manufacture using modern high speed compression manufacturing equipment.

Thus, in one aspect the invention is a delivery system comprising a central core, a first outer zone, and a second outer zone;

in which:

the central core comprises one or more biologically active ingredients;

the first outer zone partially surrounds the core,

the second outer zone partially surrounds the core,

at least one of the first outer zone and the second outer zone comprises one or more biologically active ingredients, which one or more biologically active ingredients are the same as or different than the one or more biologically active ingredients in the core;

the first outer zone and the second outer zone are heterogeneous with respect to each other,

the first outer zone and the second outer zone together form a continuous layer completely enclosing the core,

the first outer zone comprises a barrier suitable for timed release of biologically active ingredients;

the second outer zone comprises a barrier suitable for timed release of biologically active ingredients;

the central core, the first outer zone, and the second outer zone together comprise a biologically effective dosage amount of each of the one or more biologically active ingredients.

### BRIEF DESCRIPTION OF THE DRAWINGS

FIG. 1 is a graph that shows changes in viscosity, surface area, and drug transport into the blood as the delivery system progresses through the digestive tract.

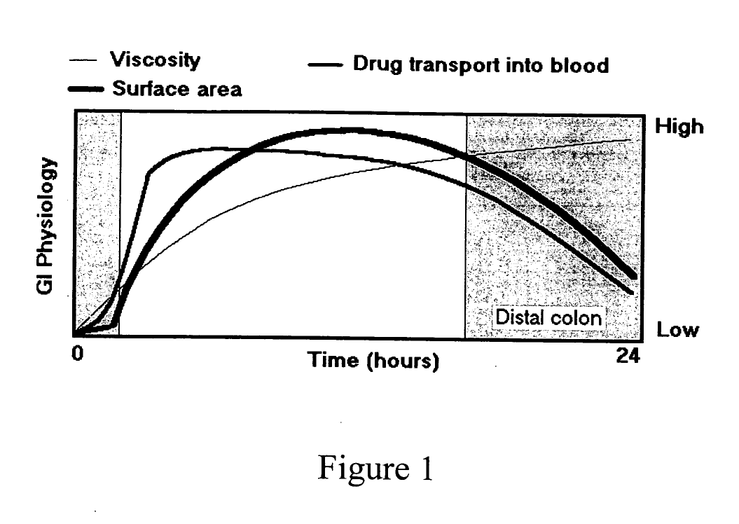

FIG. 2 is a cross-sectional view through the center of the delivery system.

FIG. 3 is a schematic showing how the invention may be used in the gastro-retentive delivery of drugs.

FIG. 4 presents graphs showing the release profiles for the delivery systems of the examples.

### DETAILED DESCRIPTION OF THE INVENTION

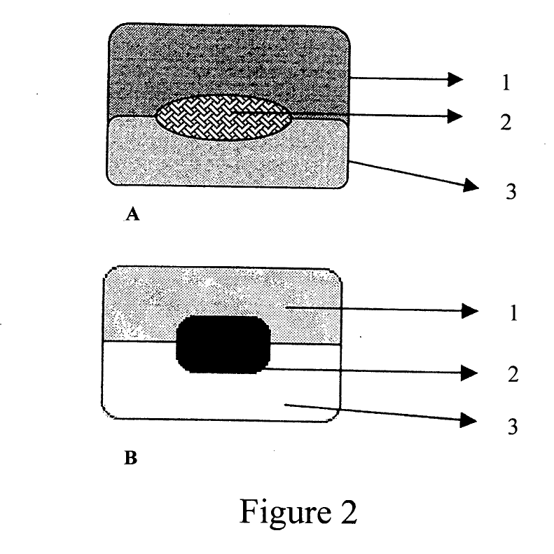

Referring to FIG. 2, the delivery system A and B is a coated tablet comprising a minimum of three regions, a first outer zone (zone A) and a second outer zone (zone B) 1 and 3, and a central core (C) 2. Zone A, is a first outer partial layer, partially surrounding the core, and comprises a barrier suitable for timed release of biologically active ingredient(s) from zone A, the core, or both zone A and the core. Zone B is a second outer partial layer, partially surrounding the core, and comprises a barrier suitable for timed release of biologically active ingredient(s) from zone B, the core, or both zone B and the core. Zones A and B are heterogeneous with respect to each other and together form a continuous heterogeneous layer fully surrounding the core.

Zones A and B may be symmetric or asymmetric with respect to each other. The term “asymmetric” refers to the weight or volume of zones A and B with respect to each other. These zones are asymmetric when they are present in a weight or volume ratio substantially above or below about 50:50, for example 60:40, as shown in FIG. 2A, and are symmetrical when their volumes or weights (depending on how measured) are substantially equal, i.e. they are present in a weight or volume ratio of about 50:50, as shown in FIG. 2B. When zones A and B are asymmetric, one of zones A or B may surround a larger volume of the core than the other of zones A or B.

Zones A and B are “heterogeneous” with respect to each other when the make-up or composition of one differs from the make-up or composition of the other. The differences in heterogeneity may reside in the nature and/or amounts of the barrier or barriers used in zone A and zone B. That is the zones may be heterogeneous with respect to their chemical content. Alternatively, or additionally, the zones may differ in the nature and/or amounts of the biologically active ingredient or ingredients contained in zone A and in zone B. That is, the zones may be heterogeneous with respect to their biologically active ingredient content. The biologically active ingredient content of a zone or the core includes the nature, amount, and number of biologically active ingredients present in that region of the delivery system.

The core and at least one of zone A and zone B together comprise a biologically effective dosage amount of at least one biologically active ingredient. Provided the zones are heterogeneous, the biologically active ingredient or ingredients contained in each of these three regions of the delivery system may be the same or different, and the amount of each ingredient contained in each of these three regions of the delivery system may be the same or different. “Biologically active ingredient” or “active ingredient” refers to any compound, composition of matter or mixture thereof which provides some physiological, psychological, biological, or pharmacological, and often beneficial, effect when administered to a subject. Typically, biologically active ingredients include drugs, including, but not limited to, those mentioned herein. A biologically effective dosage amount is the amount required to provide a physiological, psychological, biological, or pharmacological, and often beneficial, effect when administered to a subject. As will be apparent to those skilled in the art, a biologically effective dosage amount will depend, for example, on the biologically active ingredient administered; the species (human or other animal) of the subject; the sex, age, and medical condition of the subject; as well as on the nature and magnitude of the desired effect.

Zone A and zone B each comprise a barrier, which is suitable for timed release of at least one of the one or more biologically active ingredients. The barrier may comprise one or more hydrophilic or hydrophobic polymers, or other hydrophobic materials as described below. The polymer content of zone A may differ from the polymer content of zone B in either the type of polymer or amount of polymer. For example, the polymer in either zone A or zone B may be pH sensitive, so that the zone remains intact in the acidic environment of the stomach (protecting either the drug from this environment or the stomach from the drug), but dissolves in the more alkaline environment of the intestine.

In one embodiment, zone A may contain a polymer which erodes at a faster rate than the polymer in zone B, effecting different release rates for the active ingredients in zones A and B. In a different embodiment, zone A may contain a different active ingredient than zone B or the core. Alternatively, zone A may contain a different active ingredient than zone B, but the same active ingredient as the core, a different amount of the same active ingredient in zone B or the core, or either zone A or zone B may contain no active ingredient.

Zones A and B may each comprise a polymer suitable for independently controlling the timing and rate of release of a biologically active ingredient or ingredients contained in either or both such zones, together with conventional additives suitable for facilitating tablet processing or compression, for example, flow aids such as lactose, microcrystalline cellulose, cyclodextrins, adipic acid, sodium deoxycholate, and polysaccharides; lubricants such as magnesium stearate, hydrogenated vegetable oil, sodium stearyl fumarate; colorants; binders such as hydroxypropyl methyl cellulose (HPMC) and carboxymethyl cellulose, and other conventional excipients, all of which are well known to those skilled in the tablet processing art.

Polymers suitable for use in one or both of zones A and B may be swellable or nonswellable, and include, for example, hydrophilic polymers comprising celluloses such as hydroxyproplymethylcellulose, hydroxypropylcellulose, sodium carboxymethylcellulose, carboxymethylcellulose calcium, and methylcellulose; polyethylene oxide; alginates such as sodium alginate, ammonium alginate, potassium alginate, calcium alginate, propylene glycol alginate, and alginic acid; other polysaccharides such as potassium pectate, potassium pectinate, calcium pectinate, pectin, guar gum, xanthan gum, karaya gum, gum arabic, gum tragacanth, locust bean gum, agar, carrageenan, and konjac; polyvinyl alcohol; povidone; and carbomer. Suitable hydrophobic polymers include celluloses such as ethyl cellulose, hydroxyethylcellulose; cellulose acetate, cellulose acetate butyrate, cellulose acetate phthalate; methacrylic acid derivatives such as ammonio methacrylate copolymer (EUDRAGIT® RL or EUDRAGIT® RS), methacrylic acid copolymers (EUDRAGIT® L or EUDRAGIT® S), methacrylic acid-acrylic acid ethyl ester copolymer (EUDRAGIT® L 100-5), methacrylic acid ester neutral copolymer (EUDRAGIT® NE30D), dimethylaminoethylmethacrylate-methacrylic acid ester copolymer (EUDRAGIT® E 100), vinyl methyl ether/maleic anhydride copolymers, their salts and esters (GANTREZ®). Other hydrophobic materials which may be used include waxes such as beeswax, carnauba wax, microcrystalline wax, and ozokerite; fatty alcohols such as cetostearyl alcohol, stearyl alcohol, cetyl alcohol and myristyl alcohol; and fatty acid esters such as glyceryl monostearate, glycerol monooleate, acetylated monoglycerides, tristearin, tripalmitin, cetyl esters, wax, glyceryl palmitostearate, glyceryl behenate, chitosan and hydrogenated castor oil. Other representative polymeric materials suitable for compression tableting include poly(olefin), poly(vinyl), poly(carbohydrate), poly(peptides), poly(condensation), poly(rubber), poly(silicon), poly(ethylene), poly(propylene), copoly(ethylenevinylacetate), poly(isobutylethylene), poly(vinylacetate), poly(isobutylethylene), poly(vinylacetate), cross-linked poly(vinyl-alcohol), poly(methyacrylate), poly(amide), poly(ester), poly(ether), and poly(silicone) resins. Those skilled in the art will recognize that these are representative, and not exclusive, listings and that other polymeric compounds will also be suitable.

The choice of polymer used in each zone is dependent on the solubility characteristics of the biologically active ingredient contained in each of zones A and B and by the release characteristics to be achieved by each of these zones. As is well known to those skilled in the art, the release characteristics of a barrier depend on, for example, the molecular weight, solubilizing rate, swelling rate, and/or permeability of the barrier polymer. These parameters may in turn may depend on the pH, moisture and temperature of the environment as well as on the size, shape and thickness of the barrier.

For example, with respect to highly soluble biologically active ingredients such as diltiazem, metoprolol, metformine hydrochloride, topiramate, prodrug doxifluridine, propranolol, verapamil, theophylline, paracetamol, pseudoephedrine, and niacin, one may suitably use high molecular weight hydroxyproplylmethyl cellulose (HPMC) or polyethylene oxide (PEO) in one of zones A and B to provide an initial burst of active ingredient to promptly obtain therapeutic blood levels while the delivery system is still in the stomach. To maintain the blood levels so established, a second polymer, for example ethyl cellulose or cellulose acetate, may be used in the other of zone A and B which delays release of the active ingredient contained in that zone and releases it at a rate sufficient to replace the active ingredient in the blood stream as it is metabolized and thus maintain blood levels at or above a therapeutic level as the tablet travels through the small intestine where absorption is high and nears and/or enters the distal colon where absorption is limited. Thus, in this example, the delivery system contains two biologically active ingredients, each of which is released over a time and at a rate which establishes or maintains at least the minimal therapeutic blood level for each active ingredient over an extended period of time in accordance with a scheduled dosage regimen.

Conversely, when the active ingredient is relatively less soluble and/or difficult to absorb, for example acyclovir, neomycin B, captopril, cimetidine, ranitidine, enalaprilate, alendronate, atenolol, danazol, ketoconazole, mefenamic acid, nisoldipine, nifedipine, nicardipine, felodipine, atovaquone, griseofulvin, troglitazone, glibenclamide, carbamazepine, it can be solubilized with the aid of granulation, or inclusion of agents, such as surfactants, solubilizers, pH modifiers, salts, fatty acid derivatives, nonoparticles, dispersed drugs and liposomes, in zone A, zone B, or the core.

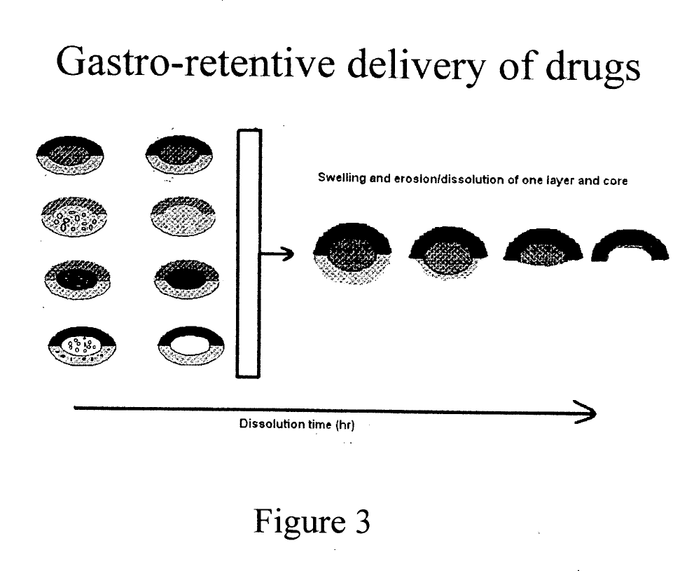

In one preferred embodiment shown in FIG. 3, either zone A or zone B may comprise a substantially non-erodable, swellable zone containing a gas generating material or materials, which contains no active ingredients and does not erode, but remains throughout the life of the delivery system. As the erodable zone erodes and releases its active ingredient, the non-erodable zone retards release of the active ingredient in the core. FIG. 3 also shows that an active ingredient in the core and the erodable zone may be powdered or granulated to further regulate its release rate. Those skilled in the art will appreciate that limited erosion will occur in the “non-erodable” zone.

Gas generation in the non-erodable zone enhances floatation of the delivery system in the gastrointestinal tract, particularly in the stomach. The gas evolving material is used to evolve gas which will cause the delivery system to float, and increase the time of retention of the delivery system in the stomach. This prevents premature passage of the delivery system into the small intestine. This is especially important for biologically active ingredients that are particularly effective from the stomach or such acidic environment.

The gas evolving material can be any conventional gas evolving system, for example, carbonates, such as sodium carbonate, sodium bicarbonate, potassium carbonate, potassium bicarbonate, and calcium carbonate. Specifically, sodium bicarbonate can be used, either alone or with citric acid. The citric acid will react with sodium bicarbonate to produce gas. However, it is generally unnecessary to add citric acid because stomach acid will also react with the sodium bicarbonate to produce gas. Because the sodium bicarbonate is intimately mixed with the barrier present in the non-erodable, swellable zone, the gas evolved is held within the swollen polymeric matrix, inflating the matrix and ensuring floatation of the delivery system. It is generally necessary to include citric acid with calcium carbonate to get acceptable gas evolution. Further, both sodium and calcium carbonates may be used together, with citric acid.

While the foregoing illustrates the different rates and release patterns from zones A and B for a delivery system containing a single active ingredient, it will be appreciated, that one of the principle advantages of the delivery system of the invention is that it also provides a system for controlling the delivery of at least two active ingredients in the same manner as described above by proper selection of polymers and amounts of ingredient to be distributed in each of the zones of the delivery system, as well as in the core.

With respect to the biologically active ingredient or ingredients contained in one or both of zones A and B as well as in the core, the active ingredient may be incorporated therein in any suitable form. For example the active ingredient may be incorporated as a powder, a crystalline material, or a granule which may itself be uncoated or coated to provide further release-controlling characteristics. The active ingredient is blended with the polymer and excipients or additives of that zone to evenly distribute the active ingredient throughout the zone or core prior to tablet compression.

It is implicit in the foregoing that the core and zones A and B, taken together, comprise a biologically effective dosage amount of each active ingredient, in which the active ingredient is distributed in each of the zones and the core in quantities sufficient to achieve the desired release rates over a long period of time and accommodate chronophysiological conditions found in the gastrointestinal tract as the delivery system progresses from the stomach to the small intestine and into the large intestine.

In addition to providing a timed delivery system for early and sustained delivery of one or more active ingredients, zones A and B may also act to delay delivery of active ingredient from the core. Thus, as zones A and/or B hydrate and swell, the outer periphery thereof erodes differentially and progressively shifts towards the central core to allow release of active ingredient from the core of the delivery system. Control of the release process is therefore predominantly a function of system configuration, related swelling dynamics and associated erosion of the external zones and internal core material. Thus, controlled heterogeneous erosion in zone A, zone B, and the core allows one to control release rates over a long period of time and to provide for gastro-retentive capability to an extent not previously possible with other compressed tablet systems.

The core of the delivery system may itself be a compressed disk or compressed tablet comprising one or more active ingredients, together with conventional tableting excipients, binders and the like. Similarly it may be a casted composite film, a laminate, an enteric coated tablet, an osmotically active tablet, a bilayer or triple layer tablet, or compressed granules or pellets containing one or more active ingredients.

A wide variety of biologically active ingredients may be used in the delivery system of the invention including, but not limited to such therapeutically or biologically active materials as cimetidine, diclofenac, diltiazem, glipizide, nifedipine, metoprolol, propranolol, theophylline, verapamil, large molecular structures such as proteins or vaccines, peptides, vitamins, minerals, and many other common drug or nutritional products which are well known or may be developed in the future. Classes of drugs which are particularly suitable for the described delivery system include cardiovascular-renal drugs such as members of the clonidine family, methyl dopa, reserpine, guanethidine, minoxidil, diazoxide, captapril, enalapril, losaritan, saralasin, felodipine, amlodipine; antidiabetic drugs such as acarbose, pioglitazone, miglitol, sulfonylureas; agents used in hyperlipidemia such as fenofibrate, gemfibrozil, lovastatin, simvastatin, flurastatin, atorvastatin; antidepressants such as paroxetine, sertraline salt, fluoxetine, citalopram, fluvoxamine, bupropion; analgesics such as oxycodone and naloxone; antibiotics such as ciprofloxacin, nalidixic acid, and other quinolones and fluoro-quinolones, Methydopa, timolol succinate and maleate,sulindac, losartan salts,indinavir sulfate, metyrosine, chlorthiazide, diflunisal, alendronate salts, thiabendazol, norfloxacin, montelukast salts, trientine salts, procainamide, hydoxyurea, atrovastatin, gabapentin, gemfibrozil, fluconazole, trovafloxacin salts, doxepin salt, dofetilide, sulfasalazine, etidronate disodium, morphine sulfate, oxycodone hydrochloride and sulphate, choline magnesium trisalicylate, quinidine sulfate, ganciclovir, methocarbamol, aspirin, saquinavir, valganciclovir, colesevelam, tolcapone, capecitabine, ortistat, irbesartan, succimer, loratadine, pseudoephedrineflutamide, labetalolo, zolpidem tartarate, celecoxib, pancrelipase, soprolol, etodolac, disulfiram, amiodaron, venlafaxine hydrochloride, hydrochlorothiazide, acebutolol, glucosamine, propoxyphene, raloxifene salt, fluoxetine, cefuroxime axetil, cefixime, abacavir sulfate, bupropion, zidovudine, lamivudine, chlorpromazine, amoxicillin, clavulanate potassium, amprenavir, sevelamer hydrochloride, carbidopa, levodopa, glyburide, gatifloxacin, cefadroxil monohydrate, quinidine gluconate, sotalolo, methenamine mandelate, moxifloxacin salts, praziquantel, quetiapine fumarate, tocainide hydrochloride and other salts, clarithromycin, divalproex sodium, erythromycin, lopinavir, ritonavir, and propafenone

Zones A and B may be present in the delivery system in a weight or volume ratio in the range of 1:10 to 10:1, respectively and together may comprise at least about 8% to about 95% by weight of the delivery system. Conversely, the core of the delivery system may comprise at least about 5% to about 92% by weight of the tablet. For example, zones A and B together may comprise from about 25% to about 75% by weight of the delivery system, and the core may correspondingly comprise from about 75% to about 25% by weight of the delivery system.

While the delivery system may be coated by conventional means such as spraying, it is preferably in the form of a compression-coated tablet which may be prepared using modern high speed tableting equipment and existing technology. More specifically, a compressible mixture is separately prepared and provided for zone A and zone B. Each of these mixtures may be prepared by blending a suitable barrier, any active ingredient(s), and conventional excipients for processing or compression tableting. One of these mixtures is introduced into the bottom of a conventional tableting die. Following introduction of the first of these mixtures (A or B), the tableted core is introduced into and centered in the die. The second of the mixtures A and B is then introduced as the third component, and the three components are compressed in the usual manner, resulting in a compression-coated tablet in which the core is embedded in and fully surrounded by the layer formed from the zones A and B deposited below, above and around the core.

The delivery system is particularly suitable for orally administered multiple drug delivery or multiple rate delivery of biologically active ingredients to the gastrointestinal environment of humans or other animals. It is only necessary for the subject to swallow the delivery system. In particular, the delivery system is especially suited to deliver at least two biologically active ingredients, each of which is released over a time and at a rate which establishes or maintains at least the minimal therapeutic blood level for each active ingredient over an extended period of time in accordance with a scheduled dosage regimen.

The advantageous properties of this invention can be observed by reference to the following examples, which illustrate but do not limit the invention.

### EXAMPLES

### General Procedures

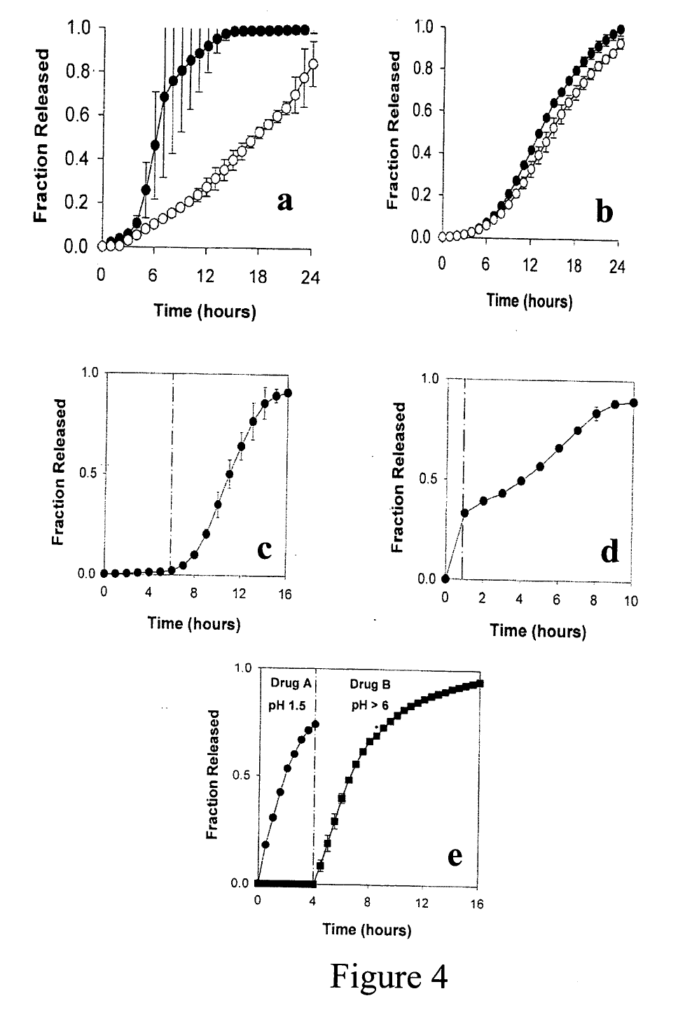

For each example, the delivery system was prepared as described above to form a compression-coated tablet. The compression-coated tablets were subjected to USP dissolution studies and drug release was determined using a spectrophotometric technique and high pressure liquid chromatography. The release profiles for each example are presented in FIG. 4.

### Example 1

Quantity (mg)

| Ingredients | Control | Invention |
|---|---|---|
| Core (Disk-Compressed Thin Slab): | | |
| Theophylline | 100 | 100 |
| Zone A: | | |
| PEO, 1 × 10⁶ MW | 75 | 75 |
| Sodium Deoxycholate | — | 12.5 |
| Adipic Acid | — | 12.5 |
| Zone B: | | |
| PEO, 1 × 10⁶ MW | 75 | 75 |
| Sodium Deoxycholate | — | 25 |

Dissolution studies were conducted with USP 23 Rotating Paddle Method (Apparatus 2), 50 rpm, buffer medium pH 1.5, 37° C. FIG. 4a shows the release profile of theophylline in buffer medium from the core, which contains no polymer, but is surrounded by two outer zones comprising polyethylene oxide (PEO) 1×10⁶ MW matrices. Solid circles represent the control and open circles represent the delivery system of the invention. The control does not include adipic acid or sodium deoxycholate. After an initial, brief lag time, theophylline release from the delivery system of the invention is linear over 24 h. In contrast, after a brief lag time, theophylline is rapidly released from the control between 3 h and 12 h.

### Example 2

Quantity (mg)

| Ingredients | Control | Invention |
|---|---|---|
| Core (Disk-Compressed Thin Slab): | | |
| Diltiazem Hydrochloride | 100 | 100 |
| Zone A: | | |
| PEO, 7 × 10⁶ MW | 200 | 200 |
| Sodium Deoxycholate | — | 12.5 |
| Adipic Acid | — | 12.5 |
| Zone B: | | |
| PEO, 7 × 10⁶ MW | 200 | 200 |
| Sodium Deoxycholate | — | 25 |

Dissolution studies were conducted with USP 23 Rotating Paddle Method (Apparatus 2), 50 rpm, buffer medium pH 1.5, 37° C. FIG. 4b shows the release profile of diltiazem hydrochloride from the core which is surrounded by two outer zones comprising PEO 7×10⁶ MW matrices. Solid circles represent the control, and open circles represent the delivery system of the invention. In Example 2, both the size and amount of polymer (PEO) is increased (compared with Example 1) in the control and the delivery system, but electrolytes are not present in the control. Diltiazem release from both the control and the delivery system follows zero-order kinetics.

### Example 3

| Ingredients | Quantity (mg) |
|---|---|
| Core: | |
| PEO, 1 × 10⁶ MW | 100 |
| Diltiazem Hydrochloride | 100 |
| Sodium Carbonate | 100 |
| Zone A: | |
| PEO, 600 000 MW | 200 |
| Zone B: | |
| PEO, 600 000 MW | 150 |
| Diltiazem Hydrochloride | 50 |

Dissolution studies were conducted with USP 23 Rotating Paddle Method (Apparatus 2), 50 rpm, buffer medium pH 1.5, 37° C. FIG. 4c shows the release profiles of diltiazem hydrochloride from a delivery system of the invention that comprises a lower MW polymer (PEO) in the two outer zones than in the core, and each outer zone has a different concentration of polymer than the other. In Example 3, the lag time prior to release is increased to approximately 6 h, and thereafter, release of diltiazem from the delivery system follows zero order kinetics.

### Example 4

| Ingredients | Quantity (mg) |
|---|---|
| Core: | |
| PEO, 1 × 10⁶ MW | 100 |
| Diltiazem Hydrochloride | 100 |
| Sodium Carbonate | 100 |
| Zone A: | |
| Microcrystalline cellulose | 150 |
| (AVICEL ® MCC) | |
| Diltiazem Hydrochloride | 50 |
| Zone B: | |
| PEO, 600 000 MW | 150 |
| Diltiazem Hydrochloride | 50 |

Dissolution studies were conducted with USP 23 Rotating Paddle Method (Apparatus 2), 50 rpm, buffer medium pH 1.5, 37° C. FIG. 4d shows the release profiles of diltiazem hydrochloride from the core, and both outer zones of the delivery system. In addition, the core comprises a PEO 1×10⁶ MW matrix, Zone A comprises microcrystalline cellulose, and Zone B comprises a PEO 600,000 MW matrix. Thus, each of the three regions of the delivery system in Example 4 contains a different type of polymer, resulting in biphasic release of diltiazem from the delivery system, i.e., an initial burst of diltiazem release followed by constant rate release.

### Example 5

| Ingredients | Quantity (mg) |
|---|---|
| Core: | |
| Enteric-coated Diclofenac Sodium Tablet | 50 |
| Zone A: | |
| PEO 600 000 MW | 100 |
| Zone B: | |
| PEO 600 000 MW | 100 |
| Cimetidine | 200 |

Dissolution studies conducted with USP 23 Rotating Paddle Method (Apparatus 2), 50 rpm, tablets moved from buffer media pH 1.5 after 4 hours to pH 6.8 for an additional 12 hours, 37° C. FIG. 4e shows the release profile of two different biologically active ingredients, cimetidine (Drug A) and diclofenac sodium (drug B), from a delivery system of the invention. Cimetidine was contained in one of the outer zones, and diclofenac in the core. Both outer zones comprised the same polymer and the core comprised no polymer. In this delivery system, release of both active ingredients followed zero order kinetics, however, cimetidine released into a buffer medium at pH 1.5, while diclofenac sodium released into a buffer medium at pH>6.

Having described the invention, we now claim the following and their equivalents.

What is claimed is:

1. A delivery system comprising a central core, a first outer zone, and a second outer zone in which:
the central core comprises one or more biologically active ingredients,
the first outer zone partially surrounds the core,
the second outer zone partially surrounds the core,
at least one of the first outer zone and the second outer zone comprises one or more biologically active ingredients, which one or more biologically active ingredients are the same as or different than the one or more biologically active ingredients in the core,
the first outer zone and the second outer zone are heterogeneous with respect to each other,
the first outer zone and the second outer zone together form a continuous layer completely enclosing the core,
the first outer zone comprises a barrier suitable for timed release of biologically active ingredients,
the second outer zone comprises a barrier suitable for timed release of biologically active ingredients, and
the core, the first outer zone, and the second outer zone together comprise a biologically effective dosage amount of each of the one or more biologically active ingredients.

2. The delivery system of claim 1 in which each of the first outer zone and the second outer zone independently releases any biologically active ingredient or ingredients contained in each zone and controls release of the active ingredient or ingredients present in the core.

3. The delivery system of claim 1 in which each of zones A and B delivers the active ingredient contained therein in different amounts, at different times, at different rates, or a combination thereof.

4. The delivery system of claim 1 in which one of the first outer zone and the second outer zone comprises at least sixty percent by weight of the combined weight of the first outer zone and the second outer zone.

5. The delivery system of claim 1 in which one of the first outer zone and the second outer zone surrounds the core to a greater extent than the other of the first outer zone and the second outer zone.

6. The delivery system of claim 1 in which the biologically active ingredient content of the first outer zone differs from the biologically active ingredient content of the second outer zone.

7. The delivery system of claim 1 in which one of the first outer zone and the second outer zone comprises a biologically active ingredient and the other of the first outer zone and the second outer zone does not comprise a biologically active ingredient.

8. The delivery system of claim 1 in which one of the first outer zone and the second outer zone comprises a first biologically active ingredient and the other of the first outer zone and the second outer zone comprises a second biologically active ingredient.

9. The delivery system of claim 1 in which the barrier of the first outer zone comprises at least one polymer and the barrier of the second outer zone comprises at least one polymer, and the first outer zone has a polymer content that differs from that of the second outer zone.

10. The delivery system of claim 1 in which the first outer zone and the second outer zone are present in a weight ratio of about 1:10 to about 10:1.

11. The delivery system of claim 1 in which the first outer zone and the second outer zone are present in a volume ratio of about 1:10 to about 10:1.

12. The delivery system of claim 1 in which the first outer zone and the second outer zone together comprise at least eight percent by weight to at least ninety-five percent by weight of the delivery system.

13. The delivery system of claim 1 in which the core is a compressed disk or tablet, a casted composite film, a laminate, an enteric coated tablet, an osmotically active tablet, a bilayer or triple layer tablet, or compressed granules, pellets or coated pellets.

14. The delivery system of claim 1 in which the core comprises about five percent to about ninety-two percent by weight of the delivery system.

15. The delivery system of claim 1 in which the core comprises from twenty-five percent to seventy-five percent by weight of the delivery system.

16. The delivery system of claim 1 in which the core, the first outer zone, and the second outer zone together comprise at least two biologically active ingredients, each of which is released over a time and at a rate which establishes or maintains at least the minimal therapeutic blood level for each active ingredient over an extended period of time in accordance with a scheduled dosage regimen.

17. A delivery system comprising a central core, a first outer zone, and a second outer zone;
in which:
the central core comprises one or more biologically active ingredients;
the first outer zone partially surrounds the core,
the second outer zone partially surrounds the core,
at least one of the first outer zone and the second outer zone comprises one or more biologically active ingredients, which one or more biologically active ingredients are the same as or different than the one or more biologically active ingredients in the core;
the first outer zone and the second outer zone are heterogeneous with respect to each other,
the first outer zone and the second outer zone together form a continuous layer completely enclosing the core,
the first outer zone comprises a barrier suitable for timed release of one or more biologically active ingredients;
the second outer zone comprises a barrier suitable for timed release of one or more biologically active ingredients;
either the barrier of the first outer zone or the barrier of the second outer zone, but not both, comprises a substantially non-erodable, swellable barrier that facilitates gastro-retentive properties of the delivery system; and
the core, the first outer zone, and the second outer zone together comprise a biologically effective dosage amount of each of the one or more biologically active ingredients;

18. The delivery system of claim 17 in which the outer zone that comprises the substantially non-erodable, swellable barrier additionally comprises a gas generating material.

19. The delivery system of claim 17 in which one of the first outer zone and the second outer zone comprises at least sixty percent by weight of the combined weight of the first outer zone and the second outer zone.

20. The delivery system of claim 17 in which one of the first outer zone and the second outer zone surrounds the core to a greater extent than the other of the first outer zone and the second outer zone.

21. The delivery system of claim 17 in which one of the first outer zone and the second outer zone comprises a biologically active ingredient and the other of the first outer zone and the second outer zone does not comprise a biologically active ingredient.

22. The delivery system of claim 17 in which the first outer zone and the second outer zone are present in a weight ratio of about 1:10 to about 10:1.

23. The delivery system of claim 17 in which the first outer zone and the second outer zone are present in a volume ratio of about 1:10 to about 10:1.

24. The delivery system of claim 17 in which the first outer zone and the second outer zone together comprise at least eight percent by weight to at least ninety-five percent by weight of the delivery system.

25. The delivery system of claim 17 in which the core is a compressed disk or tablet, a casted composite film, a laminate, an enteric coated tablet, an osmotically active tablet, a bilayer or triple layer tablet, or compressed granules, pellets or coated pellets.

26. The delivery system of claim 17 in which the core comprises about five percent by weight to about ninety-two percent by weight of the delivery system.

27. The delivery system of claim 17 in which the core comprises from twenty-five percent to seventy-five percent by weight of the delivery system.

28. The delivery system of claim 17 in which the core, the first outer zone, and the second outer zone together comprise at least two active ingredients, each of which is released over a time and at a rate which establishes or maintains at least the minimal therapeutic blood level for each active ingredient over an extended period of time in accordance with a scheduled dosage regimen.

29. A method for the controlled release of one or more biologically active ingredients, the method comprising administering a delivery system to an animal, the delivery system comprising a central core, a first outer zone, and a second outer zone,
in which:
the central core comprises one or more biologically active ingredients,
the first outer zone partially surrounds the core,
the second outer zone partially surrounds the core,
at least one of the first outer zone and the second outer zone comprises one or more biologically active ingredients, which one or more biologically active ingredients are the same as or different than the one or more biologically active ingredients in the core,
the first outer zone and the second outer zone are heterogeneous with respect to each other,
the first outer zone and the second outer zone together form a continuous layer completely enclosing the core,
the first outer zone comprises a barrier suitable for timed release of biologically active ingredients,
the second outer zone comprises a barrier suitable for timed release of biologically active ingredients, and
the core, the first outer zone, and the second outer zone together comprise a biologically effective dosage amount of each of the one or more biologically active ingredients.

30. The method of claim 29 in which the animal is a human.

31. The method of claim 30 in which at least one of the one or more biologically active ingredients is absorbed in the proximal intestine.

32. The method of claim 30 in which the one or more biologically active ingredients are selected from the group consisting of ciproflox, metformine, cyclosporine, doxiflurodine, iron salts, ampicillen, ketoconazole, micoconozole, and combinations thereof.

33. The method of claim 30 in which at least one of the one or more biologically active ingredients has high solubility in an acidic environment.

34. The method of claim 30 in which the one or more biologically active ingredients are selected from the group consisting of propranolol, metoprolol, diltiazem, verapamil, theophylline, paracetamol, pseudoephedrine sulfate, metformin hydrochloride, danazol, mefenamic acid, nisoldipine, nifedipine, nicardipine, felodipine, atovaquone, griseofulvin, troglitazone, glibenclamide, carbamazepine, acyclovir, neomycin B, captopril, enalaprilate, alendronate, atenolol, cimetidine, ranitidine, Methydopa, timolol succinate and maleate,sulindac, losartan salts, indinavir sulfate, metyrosine, chlorthiazide, diflunisal, alendronate salts, lovastatin, thiabendazol, norfloxacin, montelukast salts, trientine salts, procainamide, hydoxyurea, atrovastatin, gabapentin, gemfibrozil, fluconazole, trovafloxacin salts, doxepin salt, dofetilide, sertraline salt, sulfasalazine, etidronate disodium, morphine sulfate, oxycodone hydrochloride and sulphate, choline magnesium trisalicylate, quinidine sulfate, ganciclovir, methocarbamol, aspirin,saquinavir, valganciclovir, colesevelam, tolcapone, capecitabine, ortistat, irbesartan, succimer, loratadine, pseudoephedrineflutamide, labetalolo, zolpidem tartarate, celecoxib, pancrelipase, soprolol, etodolac, disulfiram, amiodaron, venlafaxine hydrochloride, hydrochlorothiazide, acebutolol, glucosamine, propoxyphene, raloxifene salt, fluoxetine, cefuroxime axetil, cefixime, abacavir sulfate, bupropion, zidovudine, lamivudine, chlorpromazine, amoxicillin, clavulanate potassium, amprenavir, sevelamer hydrochloride, carbidopa, levodopa, glyburide, gatifloxacin, cefadroxil monohydrate, quinidine gluconate, sotalolo, methenamine mandelate, moxifloxacin salts, praziquantel, quetiapine fumarate, tocainide hydrochloride and other salts, clarithromycin, divalproex sodium, erythromycin, lopinavir, ritonavir, propafenone, and combinations thereof.

35. The method of claim 30 in which the one or more biologically active ingredients are selected from the group consisting of peptides, proteins, and combinations thereof.

36. The method of claim 30 in which the core, the first outer zone, and the second outer zone together comprise at least two biologically active ingredients, each of which is released over a time and at a rate which establishes or maintains at least the minimal therapeutic blood level for each active ingredient over an extended period of time in accordance with a scheduled dosage regimen.
